SEQUENCE LISTING (40 sequences)

SEQ ID NO: 1 - DNA, 27 bp, Artificial Sequence
  quadruplex forming sequence
tggggagggtggggagggtggggaagg

SEQ ID NO: 2 - DNA, 37 bp, Artificial Sequence
  quadruplex forming sequence
gggggggggggggcgggggcgggggcgggggaggggt

SEQ ID NO: 3 - DNA, 58 bp, Artificial Sequence
  quadruplex forming sequence
ggggggggacgcgggagctgggggaggggcttggggccagggcggggcgcttaggggg

SEQ ID NO: 4 - DNA, 28 bp, Artificial Sequence
  quadruplex forming sequence
aggaaggggagggccggggggaggtggc

SEQ ID NO: 5 - DNA, 29 bp, Artificial Sequence
  quadruplex forming sequence
gggggcgcgcgcgcgcgcgcgcgcgcgcg

SEQ ID NO: 6 - DNA, 25 bp, Artificial Sequence
  quadruplex forming sequence
gggaggaagggggcgggagtcgggg

SEQ ID NO: 7 - DNA, 30 bp, Artificial Sequence
  quadruplex forming sequence
ggggacgcgggcgggggcggggggagggcg

SEQ ID NO: 8 - DNA, 34 bp, Artificial Sequence
  quadruplex forming sequence
gggagggagggaaggagggagggagggagggagc

SEQ ID NO: 9 - DNA, 20 bp, Artificial Sequence
  quadruplex forming sequence
gggggcggggcggggcgggg

SEQ ID NO: 10 - DNA, 27 bp, Artificial Sequence
  quadruplex forming sequence
ggaggaggaggaagaggaggaggaggc

SEQ ID NO: 11 - DNA, 12 bp, Artificial Sequence
  quadruplex forming sequence
ggaggaggagga

SEQ ID NO: 12 - DNA, 38 bp, Artificial Sequence
  quadruplex forming sequence
agagaagaggggaggaggaggaggagaggaggaggcgc

SEQ ID NO: 13 - DNA, 13 bp, Artificial Sequence
  quadruplex forming sequence
ggagggggagggg

SEQ ID NO: 14 - DNA, 28 bp, Artificial Sequence
  quadruplex forming sequence
aggagaaggaggaggtggaggaggaggg

SEQ ID NO: 15 - DNA, 32 bp, Artificial Sequence
  quadruplex forming sequence
ggaggaggaagaatgcgaggaggagggaggag

SEQ ID NO: 16 - DNA, 25 bp, Artificial Sequence
  quadruplex forming sequence
ccgaaggaggaaggaggaggagggg

SEQ ID NO: 17 - DNA, 20 bp, Artificial Sequence
  oligonucleotide
agggtggggagggtggggaa

SEQ ID NO: 18 - DNA, 20 bp, Artificial Sequence
  oligonucleotide
ttccccaccctccccaccct

SEQ ID NO: 19 - DNA, 12 bp, Artificial Sequence
  oligonucleotide
ggggttttgggg

SEQ ID NO: 20 - DNA, 15 bp, Artificial Sequence
  oligonucleotide
ggttggtgtggttgg

SEQ ID NO: 21 - DNA, 33 bp, Artificial Sequence
  oligonucleotide
tagagggggcgggggcgggggcgggggaggggt

SEQ ID NO: 22 - DNA, 20 bp, Artificial Sequence
  oligonucleotide
ggaggtggaggaggagggct

SEQ ID NO: 23 - DNA, 32 bp, Artificial Sequence
  oligonucleotide
gaggaggaggaggtcacggaggaggaggagaa

SEQ ID NO: 24 - DNA, 12 bp, Artificial Sequence
  oligonucleotide
ggaggaggagga

SEQ ID NO: 25 - DNA, 24 bp, Artificial Sequence
  oligonucleotide
ggaggaggaggaggaggaggagga

SEQ ID NO: 26 - DNA, 30 bp, Artificial Sequence
  oligonucleotide
aagagagaggggaggaggaagagaggagga

SEQ ID NO: 27 - DNA, 30 bp, Artificial Sequence
  oligonucleotide
gggagggagggaaggagggagggagggagc

SEQ ID NO: 28 - DNA, 25 bp, Artificial Sequence
  oligonucleotide
ggggaggaggaggaaggaggaagcc

SEQ ID NO: 29 - DNA, 16 bp, Artificial Sequence
  oligonucleotide
gggtgggtgggtgggt

SEQ ID NO: 30 - DNA, 17 bp, Artificial Sequence
  oligonucleotide
gtggtgggtgggtgggt

SEQ ID NO: 31 - DNA, 15 bp, Artificial Sequence
  oligonucleotide
ggttggtgtggttgg

SEQ ID NO: 32 - DNA, 12 bp, Artificial Sequence
  motif
ggaggaggagga

SEQ ID NO: 33 - DNA, 21 bp, Artificial Sequence
  oligonucleotide
cgcttgatgagtcagccggaa

SEQ ID NO: 34 - DNA, 27 bp, Artificial Sequence
  oligonucleotide
tggggagggtggggagggtggggaagg

SEQ ID NO: 35 - DNA, 27 bp, Artificial Sequence
  oligonucleotide
ccttccccaccctccccaccctcccca

SEQ ID NO: 36 - DNA, 30 bp, Artificial Sequence
  primer oligonucleotide
agctgctagccctgcgatgatttatactca

SEQ ID NO: 37 - DNA, 30 bp, Artificial Sequence
  primer oligonucleotide
atcgagatctagagcctttcagagaagcgg

SEQ ID NO: 38 - DNA, 11 bp, Artificial Sequence
  motif
ggaggaggagg

SEQ ID NO: 39 - DNA, 15 bp, Artificial Sequence
  sequence for template strand
tccaactatgtatac

SEQ ID NO: 40 - DNA, 35 bp, Artificial Sequence
  sequence for template strand
ttagcgacacgcaattgctatagtgagtcgtatta

(19) United States
(12) Patent Application Publication
(10) Pub. No.: US 2004/0115706 A1
(43) Pub. Date: Jun. 17, 2004

(54) High-throughput methods for identifying quadruplex forming nucleic acids and modulators thereof

(76) Inventors: Cheng He Jin, San Diego, CA (US); Mary Hye-Ok Chung, San Diego, CA (US); Adam Siddiqui-Jain, San Diego, CA (US); Jeffrey P. Whitten, Santee, CA (US); Thomas J. Farrell, Del Mar, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/660,897

(22) Filed: Sep. 11, 2003

Related U.S. Application Data
(60) Provisional application No. 60/410,475, filed on Sep. 12, 2002.

(51) Int. Cl. C12Q 1/68; C07H 21/04

(52) U.S. Cl. 435/6; 536/24.3

(57) ABSTRACT

Provided herein are high-throughput assays for identifying quadruplex interacting molecules. These assays comprise the steps of contacting a test molecule and a signal molecule with a quadruplex nucleic acid in a system, and detecting the signal produced by the signal molecule. Also provided are high-throughput assays for identifying quadruplex forming nucleic acids. These assays comprise the steps of contacting a signal molecule with a test nucleic acid in a system, and detecting a fluorescent signal produced by the signal molecule.

### RELATED PATENT APPLICATIONS

This patent application claims the benefit of provisional patent application No. 60/410,475 filed Sep. 12, 2002, entitled “High-throughput methods for identifying quadruplex forming nucleic acids and modulators thereof” and names Cheng He Jin et al. as inventors. This provisional patent application is hereby incorporated herein by reference in its entirety, including all drawings and cited documents.

### FIELD OF THE INVENTION

The invention relates to nucleic acids capable of forming a particular class of secondary structure known as a quadruplex and molecules that modulate their function.

### BACKGROUND

Developments in molecular biology have led to an understanding of how certain therapeutic compounds interact with molecular targets and lead to a modified physiological condition. Specificity of therapeutic compounds for their targets is derived in part from interactions between complementary structural elements in the target molecule and the therapeutic compound. A greater variety of target structural elements in the target leads to the possibility of unique and specific target/compound interactions. Because polypeptides are structurally diverse, researchers have focused on this class of targets for the design of specific therapeutic molecules.

In addition to therapeutic compounds that target polypeptides, researchers have also identified compounds that target DNA. Some of these compounds are effective anticancer agents and have led to significant increases in the survival of cancer patients. Unfortunately, however, these DNA targeting compounds do not act specifically on cancer cells and are therefore extremely toxic. Their unspecific action may be due to the fact that DNA requires the uniformity of Watson-Crick duplex structures for compactly storing information within the human genome. This uniformity of DNA structure may not offer a structurally diverse population of DNA molecules that can be specifically targeted.

Nevertheless, there are some exceptions to this structural uniformity, as certain DNA sequences can form unique secondary structures. For example, intermittent runs of guanines can form quadruplex structures, and complementary runs of cytosines can form i-motif structures. Formation of quadruplex and i-motif structures occurs when a particular region of duplex DNA transitions from Watson-Crick base pairing to single-stranded structures. While quadruplex DNA structures readily form under physiological conditions, formation of i-motif structures require acidic conditions, which makes their physiological relevance less likely, but still possible.

Quadruplex structures can vary in several different ways, including strand stoichiometry and strand orientation (see, e.g., FIG. 1). For example, interstrand quadruplex structures can form when four strands form a parallel quadruplex structure or two strands form a hairpin quadruplex structure. Previously described intramolecular quadruplexes have had a general sequence motif requirement of four runs of at least two contiguous guanines separated by an intervening sequence of at least two nucleotides (Marathias & Bolton, Biochemistry 38: 4355-4364 (1999)).

Researchers postulated that telomere DNA includes quadruplex structures and targeted these structures for the design of anticancer compounds. It was thought that sequestering the single-stranded DNA primer in a quadruplex structure would inhibit telomerase by eliminating the substrate required for its reverse transcriptase. See, e.g. Sun et al, J. Med. Chem. 40: 2113-2116 (1997). Inhibiting telomerase was thought to result in shortened telomere length, which may result in cell death, and it was postulated that cancer cells with one abnormally short telomere presumably would be more sensitive than non-cancerous cells to these telomerase inhibitors.

Sequences that potentially form quadruplexes have also been identified in transcriptional regulatory regions of oncogenes. Regulatory regions of these oncogenes include DNA sequences that can form single-stranded regions hypersensitive to nucleases. In the c-MYC promoter, for example, the regions which can form single-stranded structures bind transcription factors, such as cellular nucleic acid-binding protein (CNBP) and heterogeneous nuclear ribonucleoprotein (hnRNP), which are presumably required for transcriptional activation. Also, the interconversion between paranemic forms (e.g., unwound and non-B forms) and single stranded forms of regions in the c-MYC promoter is proposed to require NM23-H2 as an accessory factor (see FIG. 2, Postel et al., J. Bioenerg. Biomembr. 32: 277-284 (2000)). Also, researchers studying an insulin-linked polymorphic region (ILPR) in the insulin gene postulated that these regions regulate insulin expression in insulin-dependent diabetes mellitus via quadruplex structures (Lew et al., Proc. Natl. Acad. Sci. U.S.A. 97: 12508-12512 (2000)).

Some researchers have reported structural characteristics of quadruplex DNA. X-ray and crystallographic studies have been derived from the thrombin binding aptamer (TBA) and an HIV-integrase binding oligonucleotide (Schultze et al., J. Mol. Biol. 235: 1532-1547 (1994); Kelly et al., J. Mol. Biol. 256: 417-422 (1996); Jing et al., J. Biomol. Struct. Dyn. 15: 573-585 (1997); Jing et al., J. Biol. Chem. 273: 34992-34999 (1998)). Nuclear magnetic resonance structures of nucleic acids having GGA repeats also have been reported (Matsugami et al., J. Mol. Biol. 313:255-269 (2001)). In an effort to determine which quadruplex structures are relevant for regulating transcription, studies on the c-MYC regulatory region in vitro postulated that the quadruplex structure was stabilized by potassium ions in a basket conformation (Simonsson et al., Nucleic Acids Res. 26: 1167-1172 (1998)). Quadruplex structures were also probed by studying the interactions of molecules with quadruplex DNA (Han et al., J. Am. Chem. Soc. 121: 3561-3570 (1999); Arthanari et al., Nucleic Acids Research Vol. 26, No. 16: 3724-3728 (1998); Li et al., Biochemistry 35: 6911-6922 (1996); Arthanari et al., Anti-Cancer Drug Design 14: 317-326 (1999); Thomas et al., J. Phys. Chem. B. 105: 12628-12633 (2001); Anantha et al., Biochemistry Vol. 37, No. 9: 2709-2714 (1998); Lipscomb et al., Biochemistry 35: 2818-2823 (1996); and Ren et al., Biochemistry 38: 16067-16075 (1999)). Other studies probed quadruplex structure by fluorescence resonance energy transfer (Simonsson & Sjöback, J. Biol. Chem. 274: 17379-17383 (1999)).

Given the potential regulatory importance of quadruplex structures, a need exists for identifying nucleotide sequences in genomic DNA that form regulatory quadruplex structures. Further, a need exists for identifying molecules that interact specifically with quadruplex structures and modulate their biological function.

### SUMMARY

Certain regulatory regions in duplex DNA can transit into single-stranded quadruplex structures that may regulate important biological processes. It now has been discovered that quadruplex interacting molecules can be identified rapidly in high-throughput assays. These assays comprise contacting a test molecule and a signal molecule with a quadruplex nucleic acid in a system, and detecting the signal produced by the signal molecule. The signal produced by the signal molecule when the test molecule is present in the system and interacts with the quadruplex nucleic acid is different than the signal produced by the signal molecule when the test molecule is absent in the system or when the test molecule is present in the system and does not interact with the quadruplex nucleic acid. Thus, a test molecule is identified as a quadruplex interacting molecule when the signal detected in a system that includes the test molecule is different than the signal detected in a system that does not include the test molecule or includes a test molecule that does not interact with the quadruplex nucleic acid. In certain embodiments, the test molecule or the quadruplex nucleic acid is sometimes linked to a solid support. Also, the test molecule may be selected from a variety of molecules known in the art, such as organic molecules, inorganic molecules, or polypeptides. Polypeptide test molecules sometimes are linked to a phage in a phage display system or sometimes are expressed by microorgansisms that are part of an expression library.

Further, provided herein are high-throughput assays for identifying nucleic acids that form quadruplex structures. These assays comprise contacting a signal molecule with a test nucleic acid in a system, and detecting the fluorescent signal produced by the signal molecule. In these assays, the test nucleic acid is a genomic DNA fragment or complementary DNA fragment. Also, the signal emitted by the signal molecule when the test nucleic acid is present in the system and interacts with the signal molecule is different than signal produced by the signal molecule when the test nucleic is not present in the system or when the test nucleic acid does not form a quadruplex. Thus, a test nucleic acid is identified as a quadruplex forming nucleic acid when the signal detected in a system that includes the test nucleic acid is different than the signal detected in a system that does not include the test nucleic acid or includes a test nucleic acid that does not form a quadruplex.

### BRIEF DESCRIPTION OF THE DRAWINGS

FIG. 1 depicts different quadruplex conformations.

FIGS. 2A-2E show assay results for identifying quadruplex forming nucleic acids. FIG. 2A depicts fluorescent signals produced by NMM in the presence of a quadruplex forming nucleic acid (QB-1) or a nucleic acid that does not form a quadruplex (QB-2). FIG. 2B depicts signals generated by TMPyP4 in the presence of QB-1 or QB-2. FIGS. 2C and 2E show assay results for NMM-mediated identification of quadruplex forming nucleic acids, and FIG. 2D depicts assay results using TMPyP4 as a signal molecule. Table 2 depicts the nucleotide sequence for each oligonucleotide reported in FIG. 2E.

FIG. 3 depicts competition of telomestatin, QQ28, and serinodisaphyrin with NMM for nucleic acid QB-1.

FIGS. 4A and 4B show specificity of NMM (FIG. 4A) and ethidium bromide (EB, FIG. 4B) for single-stranded DNA and double-stranded DNA. FIG. 4C depicts a comparison between NMM and TMPyP4 fluorescent signals in response to double-stranded DNA and in the presence or absence of EB.

FIG. 5 depicts a process for identifying polypeptides that interact with quadruplex forming nucleic acids.

### DETAILED DESCRIPTION

Provided herein are high-throughput assays useful for identifying quadruplex forming nucleic acids and quadruplex interacting molecules. The quadruplex interacting molecules and quadruplex forming nucleic acids identified by the methods described herein can be utilized for modulating the biological activity of native quadruplex forming nucleic acids in cells. Also, the methods described herein can be utilized to identify quadruplex forming nucleotide sequences in genomic DNA and complementary DNA that were not previously reported as forming quadruplex structures, and thereby be utilized to identify new drug targets. Methods for utilizing the molecules identified by the methods provided herein are described in more detail hereafter.

Quadruplex Nucleic Acids

Quadruplex structures can form in certain purine-rich strands of nucleic acids. In the context of a duplex nucleic acid, certain purine rich strands are capable of engaging in a slow equilibrium between a typical duplex helix structure and both unwound and non-B-form regions. These unwound and non-B forms can be referred to as “paranemic structures,” and some forms are associated with sensitivity to S1 nuclease digestion, which can be referred to as “nuclease hypersensitivity elements” or “NHEs.” A quadruplex is one type of paranemic structure and certain NHEs can adopt a quadruplex structure.

As used herein, the term “quadruplex nucleic acid” and “quadruplex forming nucleic acid” refers to a nucleic acid in which a quadruplex structure may form. The entire length of the nucleic acid may participate in the quadruplex structure or a portion of the nucleic acid length may form a quadruplex structure. The term “test nucleic acid” as used herein refers to a nucleic acid that may or may not be capable of forming a quadruplex structure.

Quadruplex nucleic acids and test nucleic acids may comprise or consist of DNA (e.g., genomic DNA (gDNA) and complementary DNA (cDNA)) or RNA (e.g., mRNA, tRNA, and rRNA). In embodiments where a quadruplex nucleic acid or test nucleic acid is a gDNA or cDNA fragment, the fragment is often 50 or fewer, 100 or fewer, or 200 or fewer base pairs in length, and is sometimes about 300, about 400, about 500, about 600, about 700, about 800, about 900, about 1000, about 1100, about 1200, about 1300, or about 1400 base pairs in length. Methods for generating gDNA and cDNA fragments are well known in the art (e.g., gDNA may be fragmented by shearing methods and cDNA fragment libraries are commercially available). In embodiments where the quadruplex nucleic acid or test nucleic acid is a synthetically prepared oligonucleotide, the oligonucleotides can be about 8 to about 50 nucleotides in length, often about 8 to about 35 nucleotides in length, and sometimes from about 10 to about 25 nucleotides in length. Synthetic oligonucleotides can be synthesized using standard methods and equipment, such as by using an ABI™3900 High Throughput DNA Synthesizer, which is available from Applied Biosystems (Foster City, Calif.).

In addition, quadruplex nucleic acids and test nucleic acids may comprise or consist of analog or derivative nucleic acids, such as polyamide nucleic acids (PNA) and others exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WIPO publications WO 00/56746 and WO 01/14398, and related publications. Methods for synthesizing oligonucleotides comprising such analogs or derivatives are disclosed, for example, in the patent publications cited above, in U.S. Pat. Nos. 5,614,622; 5,739,314; 5,955,599; 5,962,674; 6,117,992; in WO 00/75372; and in related publications.

Often, a quadruplex nucleic acid or a test nucleic acid includes a nucleotide sequence that is identical to a native nucleotide sequence present in genomic DNA. For example, a quadruplex nucleic acid or a test nucleic acid may comprise or consist of a nucleotide sequence or a portion of a nucleotide sequence set forth in Table 1. The nucleotide sequences in Table 1 originate from regions in genomic DNA that regulate transcription of the c-MYC, PDGFA, PDGFB/c-sis, c-ABL, RET, BCL-2, Cyclin D1/BCL-1, K-RAS, c-MYB, HER-2/neu, EGFR, c-PIM, VAV, c-SRC and HMGA2.

TABLE 1

| Sequence | SEQ ID NO | Origin |
|---|---|---|
| TG₄AG₃TG₄AG₃TG₄AAGG | 1 | c-MYC |
| G₁₃CG₅CG₅CG₅AG₄T | 2 | PDGFA |
| G₈ACGCG₃AGCTG₅AG₃CTTG₄CCAG₃CG₄CGCTTAG₅ | 3 | PDGFB/c-sis |
| AGGAAG₄AG₃CCG₆AGGTGGC | 4 | c-ABL |
| G₅(CG₄)₃ | 5 | RET |
| G₃AGGAAG₅CG₃AGTCG₄ | 6 | BCL-2 |
| G₄ACGCG₃CG₅CG₆AG₃CG | 7 | Cyclin |
| | | D1/BCL-1 |
| (G₃A)₃AGGA(G₃A)₄GC | 8 | K-RAS |
| G₅(CG₄)₃ | 9 | H-RAS |
| (GGA)₄AGA(GGA)₃GGC | 10 | c-MYB |
| (GGA)₄ | 11 | VAV |
| AGAGAAGAGG(GGA)₅GAGGAGGAGGCGC | 12 | HMGA2 |
| GGAGGGGGAGGGG | 13 | c-PIM |
| AGGAGAA(GGA)₂GGT(GGA)₃G₃ | 14 | HER2/neu |
| (GGA)₃AGAATGCGA(GGA)₂G₃AGGAG | 15 | EGFR |
| CCGAA(GGA)₂A(GGA)₃G₄ | 16 | c-SRC |

While quadruplex forming sequences are typically identified in regulatory regions upstream of a gene (e.g., a promoter or a 5′ untranslated region (UTR)), quadruplex forming sequences also may be identified within a 3′ UTR or within an intron or exon of a gene.

A quadruplex nucleic acid or a test nucleic acid utilized in the assays described herein sometimes includes a nucleotide sequence that is similar to a native nucleotide sequence in genomic DNA. A similar nucleotide sequence may include modifications to the native sequence, such as substitutions, deletions, or insertions of one or more nucleotides. A quadruplex nucleic acid or a test nucleic acid may include a nucleotide sequence that conforms to the motif (GGA)₄ or (GGA)₃GG where G is guanine and A is adenine. Also, a quadruplex nucleic acid or a test nucleic acid may include a nucleotide sequence that conforms to the motif (GₐX_{b})_{c}Gₐ, where G is guanine; X is guanine, cytosine, adenine, or thymine; a is an integer between 2 to 10; b is an integer between 1 to 6; and c is the integer 3. Sometimes a is an integer between 2 and 6 and b is an integer between 1 and 4, and often, b is the integer 2 or 3. A quadruplex nucleic acid or a test nucleic acid may include one or more flanking nucleotides on the 5′ and/or 3′ end of the nucleotide sequence that forms the quadruplex that are not part of the quadruplex structure.

Quadruplex nucleic acids and test nucleic acids may be contacted in the system as single-stranded nucleic acids, double stranded nucleic acids, or other forms of nucleic acids (see, e.g., Ren & Chaires, Biochemistry 38: 16067-16075 (1999)). Double stranded nucleic acids may be presented in the system by a plasmid, as exemplified herein.

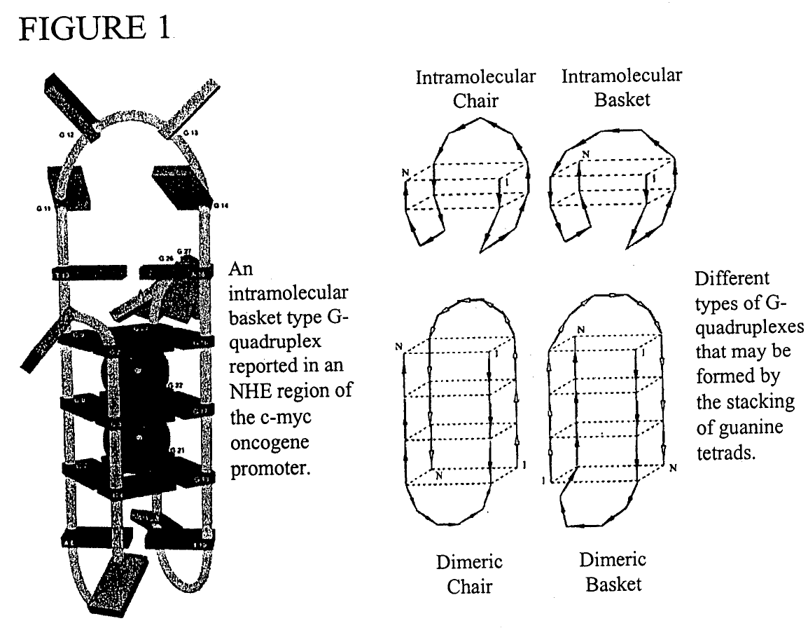

Quadruplex nucleic acids can exist in different conformations, which differ in strand stoichiometry and/or strand orientation. FIG. 1 illustrates examples of different interstrand and intrastrand quadruplex structures. The ability of guanine rich nucleic acids of adopting these structural conformations is due to the formation of guanine tetrads through Hoogsteen hydrogen bonds. Thus, one nucleic acid sequence can give rise to different quadruplex orientations, where the different conformations depend upon conditions under which they form, such as the concentration of potassium ions present in the system and the time that the quadruplex is allowed to form.

Different quadruplex conformations can be separately identified from one another using standard procedures known in the art, and as described herein. Also, multiple conformations can be in equilibrium with one another, and can be in equilibrium with duplex nucleic acid if a complementary strand exists in the system. The equilibrium may be shifted to favor one conformation over another such that the favored conformation is present in a higher concentration or fraction over the other conformation or other conformations. The term “favor” as used herein refers to one conformation being at a higher concentration or fraction relative to other conformations, which is also referred to as stabilizing the particular quadruplex conformation. The term “hinder” as used herein refers to one conformation being at a lower concentration. One conformation may be favored over another conformation if it is present in the system at a fraction of 50% or greater, 75% or greater, or 80% or greater or 90% or greater with respect to another conformation (e.g., another quadruplex conformation, another paranemic conformation, or a duplex conformation). Conversely, one conformation may be hindered if it is present in the system at a fraction of 50% or less, 25% or less, or 20% or less and 10% or less, with respect to another conformation.

Equilibrium may be shifted to favor one quadruplex form over another. For example, certain bases in a quadruplex nucleic acid may be mutated to prevent the formation of one conformation. Typically, these mutations are located in tetrad regions of the quadruplex (regions in which four bases interact with one another in a planar orientation). Also, ion concentrations and the time with which a quadruplex nucleic acid is contacted with certain ions can favor one conformation over another. For example, potassium ions stabilize quadruplex structures, and higher concentrations of potassium ions and longer contact times of potassium ions with a quadruplex nucleic acid can favor one conformation over another. The quadruplex conformation can be favored with contact times of 5 minutes or less in solutions containing 100 mM potassium ions, and often 10 minutes or less, 20 minutes or less, 30 minutes or less, and 40 minutes or less. Potassium ion concentration and the counter anion can vary, and the skilled artisan can routinely determine which quadruplex conformation exists for a given set of conditions by utilizing the methods described herein. Furthermore, different quadruplex structures may be distinguished by probing them with molecules that favorably interact with one quadruplex form over another.

Signal Molecules

Molecules that emit a detectable signal when they interact with a quadruplex nucleic acid are utilized in the assays described herein. Using the procedure set forth in Example 1, NMM was identified as being an appropriate signal molecule for the assays as it emitted a signal that scaled directly in a range of increasing quadruplex nucleic acid concentrations. Also, NMM emitted a signal that did not change significantly with increasing concentrations of a nucleic acid incapable of forming a quadruplex (e.g., the signal intensity typically varies by 10% or less or 5% or less, and sometimes 15% or less or 20% or less). The procedure set forth in Example 1 can be utilized routinely for screening signal molecules appropriate for the assays described herein.

As used herein, the term “scales directly” refers to a signal that iteratively increases or decreases in response to a range of increasing quadruplex nucleic acid concentrations. Appropriate signal molecules typically exhibit a hyperbolic relationship when signal intensity is plotted as a function of quadruplex nucleic acid concentration. Inappropriate signal molecules do not emit a signal that scales directly with quadruplex nucleic acid concentration in any concentration range. Inappropriate signal molecules may also emit a signal that varies significantly when contacted with a nucleic acid incapable of forming a quadruplex (e.g., the intensity of the signal varies by 50% or more, and sometimes 25% or more or 40% or more).

As used herein, the term “interacts” typically refers to reversible binding of a signal molecule and/or test molecule to a quadruplex nucleic acid or test nucleic acid. Interactions between signal molecules and nucleic acids or interactions between test molecules and nucleic acids can be quantified. Often, binding affinity is quantified by plotting signal intensity as a function of a range of signal molecule concentrations, test molecule concentrations, and/or nucleic acid concentrations. Quantified interactions can be expressed in terms of a concentration of signal molecule, test molecule, or nucleic acid required for emission of a signal that is 50% of the maximum signal (IC₅₀). Also, quantified interactions can be expressed as a dissociation constant (K_{d} or Kᵢ) using kinetic methods known in the art.

A variety of signals can be detected in the assays described herein. A fluorescence signal is typically monitored in the assays by exciting a fluorophore at a specific excitation wavelength and then detecting fluorescence emitted by the fluorophore at a different emission wavelength. Many nucleic acid interacting fluorophores and their attendant excitation and emission wavelengths are known in the art (Anantha et al., Biochemistry 37: 2709-2714 (1998); Qu & Chaires, Methods Enzymol 321:353-69 (2000)). Standard methods for detecting fluorescent signals are also known in the art, such as by using the detector referenced in Example 1. Background fluorescence may be reduced in the system with the addition of photon reducing agents (see, e.g., U.S. Pat. No. 6,221,612), which can enhance the signal to noise ratio.

Another signal that can be detected is a change in refractive index at a solid optical surface, where the change is caused by the binding or release of a refractive index enhancing molecule near or at the optical surface. These methods for determining refractive index changes of an optical surface are based upon surface plasmon resonance (SPR). SPR is observed as a dip in light intensity reflected at a specific angle from the interface between an optically transparent material (e.g., glass) and a thin metal film (e.g., silver or gold). SPR depends upon the refractive index of the medium (e.g., a sample solution) close to the metal surface. A change of refractive index at the metal surface, such as by the adsorption or binding of material near the surface, will cause a corresponding shift in the angle at which SPR occurs. SPR signals and uses thereof are further exemplified in U.S. Pat. Nos. 5,641,640; 5,955,729; 6,127,183; 6,143,574; and 6,207,381, and WIPO publication WO 90/05295 and apparatuses for measuring SPR signals are commercially available (Biacore, Inc., Piscataway, N.J.). In one embodiment, a molecule that interacts with a quadruplex nucleic acid can be linked via a linker to a chip having an optically transparent material and a thin metal film, and interactions between the molecule and the nucleic acid can be detected by changes in refractive index.

Other signals representative of structure may also be detected, such as NMR spectral shifts (see, e.g., Arthanari & Bolton, Anti-Cancer Drug Design 14: 317-326 (1999)) and fluorescence resonance energy transfers (see, e.g., Simonsson & Sjöback, J. Biol. Chem. 274: 17379-17383 (1999)). In embodiments where a quadruplex nucleic acid or test nucleic acid is attached to a solid support, assays may employ other types of signal molecules, where unbound signal molecule can be separated from signal molecule bound to the nucleic acid. For example, a signal molecule may be labeled with a radioactive isotope (e.g., ¹²⁵I, ¹³¹I, ³⁵S, ³²P, ¹⁴C or ³H); a light scattering label (Genicon Sciences Corporation, San Diego, Calif. and see, e.g., U.S. Pat. No. 6,214,560); an enzymic or protein label (e.g., GFP or peroxidase); or another chromogenic label or dye (e.g., Texas Red).

Test Molecules

One or more test molecules may be added to a system in assays for identifying quadruplex interacting molecules. Test molecules, signal molecules, and nucleic acids can be added to the system in any order. For example, a test molecule may be added to a system after a signal molecule and/or a nucleic acid are added; a test molecule may be added to a system before a signal molecule and/or a nucleic acid are added; or a test molecule may be added simultaneously to a system with a signal molecule and/or a nucleic acid. Nucleic acids and test molecules often are added to a system and then the signal molecule is added.

As noted above, test molecules and nucleic acids typically interact by reversible binding. Often, the presence of quadruplex interacting molecules with a quadruplex nucleic acid and a signal molecule decreases the signal emitted by the signal molecule in comparison to the signal intensity emitted when no quadruplex interacting molecule is present in the system. Also, the signal often scales directly with a range of increasing quadruplex interacting molecule concentrations. Like appropriate signal molecules for the assay, quadruplex interacting molecules often exhibit a hyperbolic relationship when signal intensity is plotted as a function of quadruplex interacting molecule concentration. Sometimes, the quadruplex interacting molecule increases the signal emitted by the signal molecule where the signal molecule emits a signal that decreases with increasing quadruplex nucleic acid concentrations. In addition to reversible binding, test molecules may interact with nucleic acids with irreversible binding, by cleaving one or more strands of a nucleic acid, or by adding chemical moieties to the nucleic acid (e.g., alkylation), for example, depending upon the structure and function of the test molecule.

Test molecules often are organic or inorganic compounds having a molecular weight of 10,000 grams per mole or less, and sometimes having a molecular weight of 5,000 grams per mole or less, 1,000 grams per mole or less, or 500 grams per mole or less. Also included are salts, esters, and other pharmaceutically acceptable forms of the compounds. Compounds that interact with nucleic acids are known in the art (see, e.g. Hurley, Nature Rev. Cancer 2, 188-200 (2002); Anantha., Biochemistry Vol. 37, No. 9: 2709-2714 (1998); and Ren et al., Biochemistry 38: 16067-16075 (1999)).

Compounds can be obtained using any of the combinatorial library methods known in the art, including spatially addressable parallel solid phase or solution phase libraries; synthetic library methods requiring deconvolution; “one-bead one-compound” library methods; and synthetic library methods using affinity chromatography selection. Examples of methods for synthesizing molecular libraries are described, for example, in DeWitt et al., Proc. Natl. Acad. Sci. U.S.A. 90: 6909 (1993); Erb et al., Proc. Natl. Acad Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and Gallop et al., J. Med. Chem. 37: 1233 (1994).

In addition to an organic and inorganic compound, a test molecule is sometimes a nucleic acid, a catalytic nucleic acid (e.g., a ribozyme), a nucleotide, a nucleotide analog, a polypeptide, an antibody, or a peptide mimetic. Methods for making and using these test molecules are known in the art. For example, methods for making ribozymes and assessing ribozyme activity are described (see, e.g., U.S. Pat. Nos. 5,093,246; 4,987,071; and 5,116,742; Haselhoff & Gerlach, Nature 334: 585-591 (1988) and Bartel & Szostak, Science 261: 1411-1418 (1993)). Also, peptide mimetic libraries are described (see, e.g., Zuckermann et al., J. Med. Chem. 37: 2678-85 (1994)).

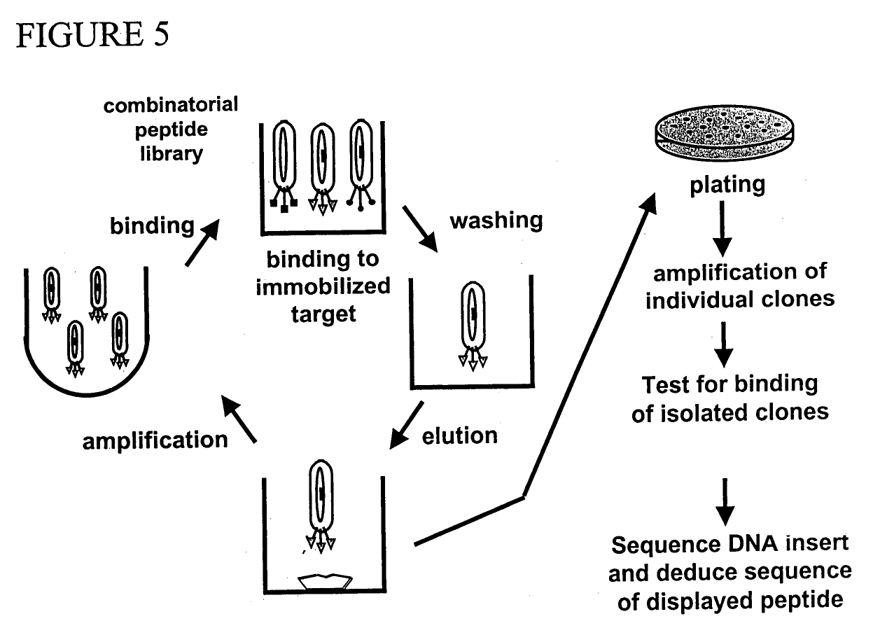

Polypeptide test molecules can be added to the system in free form or may be linked to a solid support or another molecule. For example, polypeptide test molecules may be linked to a phage via a phage coat protein. The latter embodiment is often accomplished by using a phage display system, where quadruplex nucleic acids linked to a solid support are contacted with phages that display different polypeptide test molecules. Phages displaying polypeptide test molecules that interact with the immobilized nucleic acids adhere to the solid support, and phage nucleic acids corresponding to the adhered phages are then isolated and sequenced to determine the sequence of the polypeptide test molecules that interacted with the immobilized nucleic acids. A schematic for this process is set forth in FIG. 5 and a specific embodiment of the process is set forth in Example 4.

Methods for displaying a wide variety of peptides or proteins as fusions with bacteriophage coat proteins are well known (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991)). Methods are also available for linking the test polypeptide to the N-terminus or the C-terminus of the phage coat protein. The original phage display system was disclosed, for example, in U.S. Pat. Nos. 5,096,815 and 5,198,346. This system used the filamentous phage M13, which required that the cloned protein be generated in E. coli and required translocation of the cloned protein across the E. coli inner membrane. Lytic bacteriophage vectors, such as lambda, T4 and T7 are more practical since they are independent of E. coli secretion. T7 is commercially available and described in U.S. Pat. Nos. 5,223,409; 5,403,484; 5,571,698; and 5,766,905.

Systems and Solid Supports

In the assays, signal molecules and/or test molecules are contacted with a nucleic acid in a system. As used herein, the term “contacting” refers to placing a signal molecule and/or a test molecule in close proximity to a quadruplex nucleic acid or test nucleic acid and allowing the molecules to collide with one another by diffusion. Contacting these assay components with one another can be accomplished by adding assay components to one body of fluid or in one reaction vessel, for example. The components in the system may be mixed in variety of manners, such as by oscillating a vessel, subjecting a vessel to a vortex generating apparatus, repeated mixing with a pipette or pipettes, or by passing fluid containing one assay component over a surface having another assay component immobilized thereon, for example.

As used herein, the term “system” refers to an environment that receives the assay components, which includes, for example, microtitre plates (e.g., 96-well or 384-well plates), silicon chips having molecules immobilized thereon and optionally oriented in an array (see, e.g., U.S. Pat. No. 6,261,776 and Fodor, Nature 364: 555-556 (1993)), and microfluidic devices (see, e.g., U.S. Pat. Nos. 6,440,722; 6,429,025; 6,379,974; and 6,316,781). The system can include attendant equipment for carrying out the assays, such as signal detectors, robotic platforms, and pipette dispensers.

One or more assay components may be immobilized to a solid support. The attachment between an assay component and the solid support may be covalent or non-covalent (see, e.g., U.S. Pat. No. 6,022,688 for non-covalent attachments). The solid support may be one or more surfaces of the system, such as one or more surfaces in each well of a microtiter plate, a surface of a silicon wafer, a surface of a bead (see, e.g., Lam, Nature 354: 82-84 (1991)) that is optionally linked to another solid support, or a channel in a microfluidic device, for example. Types of solid supports, linker molecules for covalent and non-covalent attachments to solid supports, and methods for immobilizing nucleic acids and other molecules to solid supports are well known (see, e.g., U.S. Pat. Nos. 6,261,776; 5,900,481; 6,133,436; and 6,022,688; and WIPO publication WO 01/18234).

Identifying Quadruplex Nucleic Acids and Quadruplex Interacting Molecules

Test molecules often are identified as quadruplex interacting molecules where the signal produced by the signal molecule in a system containing the test molecule is different than the signal produced by the signal molecule in a system not containing the test molecule. Also, test nucleic acids are identified as quadruplex forming nucleic acids when the signal detected in a system that includes the test nucleic acid is different than the signal detected in a system that does not include the test nucleic acid. While background signals may be assessed each time a new test molecule or test nucleic acid is probed by the assay, detecting the background signal is not required each time a new test molecule or test nucleic acid is assayed.

In addition to determining whether a test molecule or test nucleic acid gives rise to a different signal, the affinity of the interaction between the nucleic acid and test molecule or signal molecule may be quantified as described previously. IC₅₀, K_{d}, or Kᵢ threshold values may then be compared to the measured IC₅₀ or K_{d} values for each interaction, and thereby identify a test molecule as a quadruplex interacting molecule or a test nucleic acid as a quadruplex forming nucleic acid. For example, IC₅₀ or K_{d} threshold values of 10 μM or less, 1μM or less, and 100 nM or less are often utilized, and sometimes threshold values of 10 nM or less, 1 nM or less, 100 pM or less, and 10 pM or less are utilized to identify quadruplex interacting molecules and quadruplex forming nucleic acids.

Further, secondary assays can be utilized to confirm the identification of quadruplex interacting molecules and quadruplex forming nucleic acids. For example, gel mobility shift assays (see, e.g., Jin & Pike, Mol. Endocrinol. 10: 196-205 (1996)), polymerase arrest assays, transcription reporter assays, and apoptosis assays (see, e.g., Amersham Biosciences (Piscataway, N.J.)) can be utilized. Also, topoisomerase assays can be utilized subsequently to determine whether the quadruplex interacting molecules have a topoisomerase pathway activity (see, e.g., TopoGEN, Inc. (Columbus, Ohio))

An example of an arrest assay is a system that includes a template nucleic acid, which may comprise a quadruplex forming sequence, and a primer nucleic acid which hybridizes to the template nucleic acid 5′ of the quadruplex-forming sequence. The primer is extended by a polymerase (e.g., Taq polymerase), which advances from the primer along the template nucleic acid. In this assay, a quadruplex structure can block or arrest the advance of the enzyme, leading to shorter transcription fragments. Also, the arrest assay may be conducted at a variety of temperatures, including 45° C. and 60° C., and at a variety of ion concentrations.

In a transcription reporter assay, test quadruplex DNA may be coupled to a reporter system, such that a formation or stabilization of a quadruplex structure can modulate a reporter signal. An example of such a system is a reporter expression system in which a polypeptide, such as luciferase or green fluorescent protein (GFP), is expressed by a gene operably linked to the potential quadruplex forming nucleic acid and expression of the polypeptide can be detected. As used herein, the term “operably linked” refers to a nucleotide sequence which is regulated by a sequence comprising the potential quadruplex forming nucleic acid. A sequence may be operably linked when it is on the same nucleic acid as the quadruplex DNA, or on a different nucleic acid. An exemplary luciferase reporter system is described herein.

Utilization of Molecules Identified by the High-Throughput Assays

Because quadruplex forming nucleic acids are regulators of biological processes such as oncogene transcription, modulators of quadruplex biological activity can be utilized as therapeutics. For example, molecules that interact with and stabilize quadruplex structures may exert a therapeutic effect on certain cell proliferative disorders because abnormally increased oncogene expression can cause cell proliferative disorders and quadruplex structures typically down-regulate oncogene expression. Similarly, administering a quadruplex forming nucleic acid that has a similar or identical nucleotide sequence to a native oncogene regulating quadruplex sequence may act as a decoy by competing for cellular molecules that normally up-regulate the oncogene. Thus, quadruplex forming nucleic acids and quadruplex interacting molecules identified by the methods described herein may be administered to cells, tissues, or organisms for the purpose of down-regulating oncogene transcription and thereby alleviating cell proliferative disorders.

Determining whether the biological activity of native quadruplex DNA is modulated in a cell, tissue, or organism can be accomplished by monitoring quadruplex biological activity. Quadruplex biological activity may be monitored in cells, tissues, or organisms, for example, by detecting a decrease or increase of gene transcription in response to contacting the quadruplex DNA with a molecule. Transcription can be detected by directly observing RNA transcripts or observing polypeptides translated by transcripts, which are methods well known in the art.

Quadruplex interacting molecules and quadruplex forming nucleic acids can be utilized to target many cell proliferative disorders. Cell proliferative disorders include, for example, hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol. 11:267-97 (1991)); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Also, administering a molecule to a subject that specifically interacts and stabilizes one or more quadruplexes in the HMGA2 promoter can reduce transcription of the HMGA2 gene and thereby reduce adipose cell proliferation. By reducing adipose cell proliferative disorders such as adiposity and lipomas, molecules that stabilize one or more quadruplexes in the HMGA2 promoter can reduce obesity.

Administering a molecule to an organism can be accomplished in a number of manners, including intradermal, intramuscular, intravenous, intraperitoneal, and subcutaneous administration. An effective amount of molecule for modulating the biological activity of native quadruplex DNA will depend in part on the molecule composition, the mode of administration, and the weight and general health of the organism, and can generally range from about 1.0 μg to about 5000 μg of peptide for a 70 kg patient. The effective amount can be optimized by determining whether the biological activity of the native quadruplex DNA is modulated in the system.

Thus, provided herein are methods for reducing cell proliferation or for treating or alleviating cell proliferative disorders, which comprise contacting a system having a native quadruplex DNA with a quadruplex interacting molecule or quadruplex forming nucleic acid identified by an assay described herein. The system sometimes is a group of cells or one or more tissues, and often is a subject in need of a treatment of a cell proliferative disorder (e.g., a mammal such as a mouse, rat, monkey, or human).

### EXAMPLES

The invention is further illustrated by the following examples, which should not be construed as limiting.

### Example 1

### Selection of Signal Molecules

The following assay was utilized to identify signal molecules that generated signals which scaled directly with the interaction of the signal molecule with quadruplex DNA. This assay was useful for determining whether a particular signal molecule was appropriate for the assays described herein.

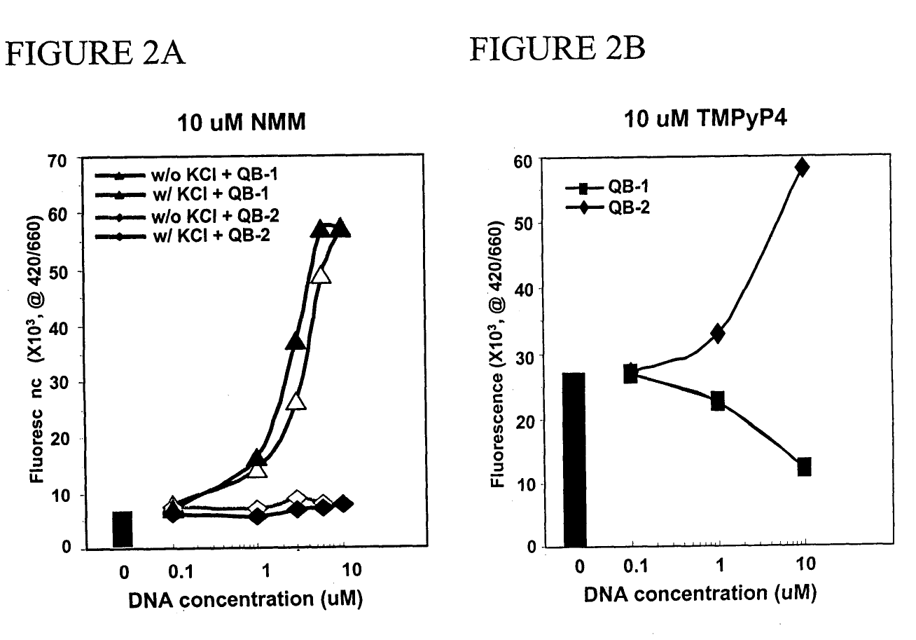

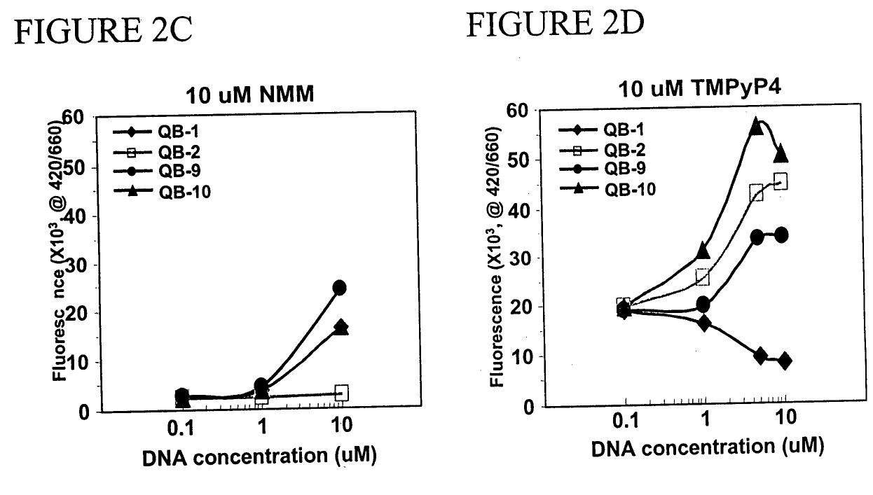

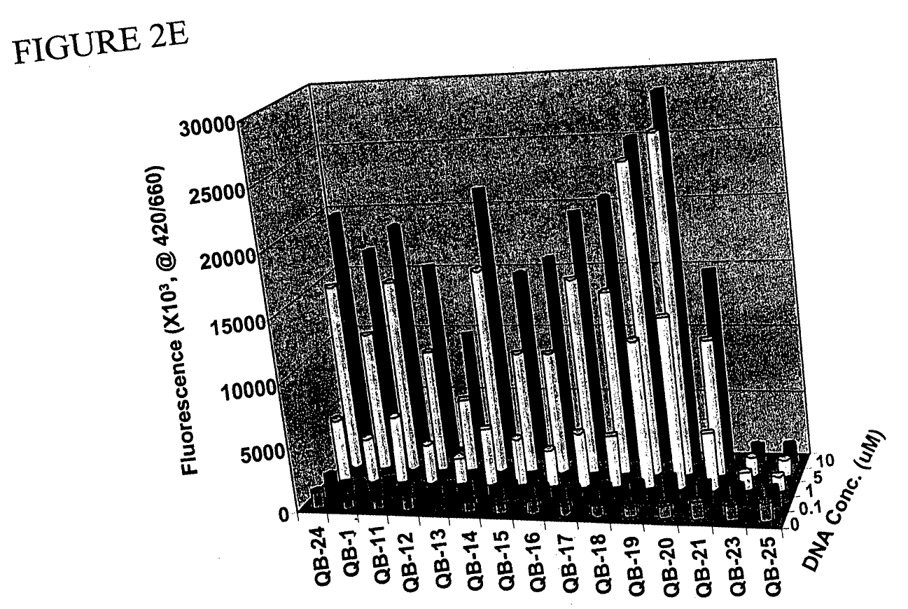

A typical assay was carried out in 100 μl of 20 mM HEPES buffer, pH 7.0, 140 mM NaCl, and 100 mM KCl. Fifty μl of quadruplex nucleic acid or a nucleic acid not capable of forming a quadruplex were added in 96-well plate. 50 μl of signal molecule was then added for a final concentration of 3 μM. N-methylmesoporphyrin IX (NMM) and meso-tetrakis(N-methyl-4-pyridyl)porphine (TMPyP4) were tested as signal molecules. Fluorescence was measured at an excitation wavelength of 420 nm and an emission wavelength of 660 nm for each of these signal molecules using a FluoStar 2000 fluorometer (BMG Labtechnologies, Durham, N.C.). NMM and TMPyP4 were obtained from Frontier Scientific Inc, Logan, Utah. Fluorescence was plotted as a function of concentration of nucleic acid added to the system at a constant 3 μM concentration of NMM (FIG. 2A) or TMPyP4 (FIG. 2B).

FIG. 2A demonstrated that NMM interacted with quadruplex nucleic acid QB-1 and did not interact appreciably with non-quadruplex nucleic acid QB-2. Also, FIG. 2A showed that NMM could stabilize quadruplex structure without the addition of potassium ions. FIG. 2B demonstrated that TMPyP4 produced a signal that varied significantly with both quadruplex and non-quadruplex nucleic acids. Also, FIG. 2B demonstrated that TMPyP4 emitted a signal that decreased with increasing quadruplex nucleic acid concentrations, likely due to quenching.

FIG. 2A demonstrated that NMM was a suitable signal molecule for identifying quadruplex forming nucleic acids and quadruplex interacting molecules as the fluorescent signal generated by the molecule scaled directly with increasing quadruplex nucleic acid concentrations and did not vary significantly with increasing non-quadruplex nucleic acid concentrations.

### Example 2

### Identification of Quadruplex Forming Nucleic Acids

In this study, several oligonucleotides were analyzed to determine which of them were capable of forming quadruplex structures. Assays described in Example 1 were carried out using the test nucleic acids set forth in Table 2. The oligonucleotides in Table 2 were synthesized by Applied Biosystems (Foster City, Calif.).

TABLE 2

| | | | Origin or |
|---|---|---|---|
| Sequence | Identifier | SEQ ID NO | Comments |
| 5′-AGGGTGGGGAGGGTGGGGAA-3′ | QB-1 | 17 | c-MYC |
| 5′-TTCCCCACCCTCCCCACCCT-3′ | QB-2 | 18 | c-MYC |
| 5′-GGGGTTTTGGGG-3′ | QB-9 | 19 | Dimers for |
| | | | quadruplex of |
| | | | basket or crossover |
| | | | structure |
| 5′-GGTTGGTGTGGTTGG-3′ | QB-10 | 20 | Intramolecular |
| | | | edge, or chair |
| | | | structure |
| 5′-TAGAGGGGGCGGGGGCGGGGGCGGGGGAGGGGT-3′ | QB-11 | 21 | PDGF-A |
| 5′-GGAGGTGGAGGAGGAGGGCT-3′ | QB-12 | 22 | HER-2/neu |
| 5′-GAGGAGGAGGAGGTCACGGAGGAGGAGGAGAA-3′ | QB-13 | 23 | C-MYB |
| 5′-GGAGGAGGAGGA-3′ | QB-14 | 24 | (GGA)₄ |
| 5′-GGAGGAGGAGGAGGAGGAGGAGGA-3′ | QB-15 | 25 | (GGA)₈ |
| 5′-AAGAGAGAGGGGAGGAGGAAGAGAGGAGGA-3′ | QB-16 | 26 | HMGA2 |
| 5′-GGGAGGGAGGGAAGGAGGGAGGGAGGGAGC-3′ | QB-17 | 27 | k-RAS |
| 5′-GGGGAGGAGGAGGAAGGAGGAAGCC-3′ | QB-18 | 28 | c-SRC |
| 5′-GGGTGGGTGGGTGGGT-3′ | QB-19 | 29 | T30695 |
| 5′-GTGGTGGGTGGGTGGGT-3′ | QB-20 | 30 | T30177 |
| 5′-GGTTGGTGTGGTTGG-3′ | QB-21 | 31 | TBA |
| 5′-CGCTTGATGAGTCAGCCGGAA-3′ | QB-23 | 33 | AP-1 |
| 5′-TGGGGAGGGTGGGGAGGGTGGGGAAGG-3′ | QB-24 | 34 | c-MYC |
| 5′-CCTTCCCCACCCTCCCCACCCTCCCCA-3′ | QB-25 | 35 | c-MYC |

0.1 to 10 μM of test nucleic acids QB-1, QB-2, QB-9, and QB-10 were probed with 3 μM NMM (FIG. 2C) or TMPyP4 (FIG. 2D) to identify which oligonucleotides were capable of forming a quadruplex structure. Of these oligonucleotides, QB-1, QB-9, and QB-10 were capable of forming a quadruplex structure, while QB-2 was not, according to FIG. 2C. FIG. 2E depicts quadruplex forming profiles for oligonucleotides QB-1 and QB-11 to QB-25. NMM interacted most significantly with QB-19 and QB-20 and interacted with most other oligonucleotides. Thus, FIG. 2E demonstrated that with the exception of QB-23, and QB-25, which did not appreciably interact with NMM, the remainder of the oligonucleotides assayed were quadruplex forming nucleic acids.

### Example 3

### Identification of Quadruplex Interacting Molecules

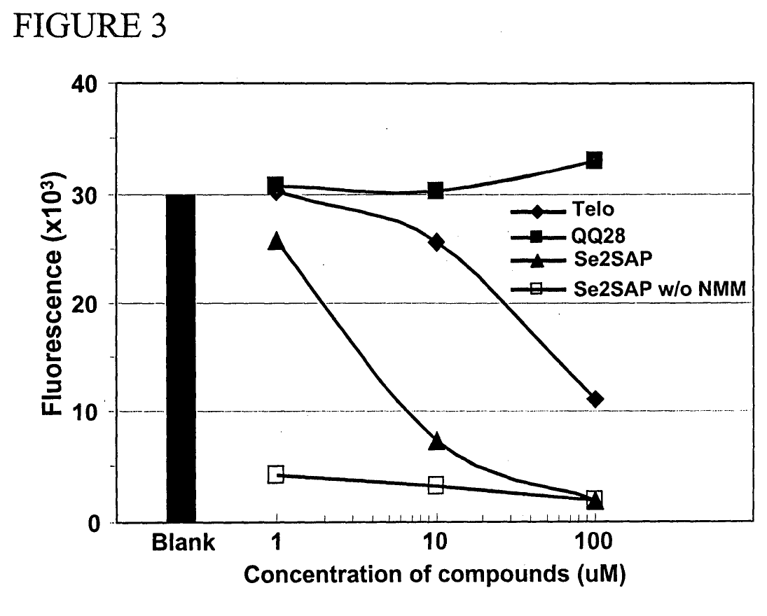

Test molecules were mixed with the quadruplex nucleic acid QB-1 in a 96-well plate and fluorescence backgrounds were monitored under the conditions specified in Example 1. NMM was then added to wells in the 96-well plate to a final concentration of 3 μM. Fluorescence of NMM was measured at an excitation wavelength of 420 nm and an emission wavelength of 660 nm using a FluoStar 2000 fluorometer (BMG Labtechnologies, Durham, N.C.). Maximum fluorescent signals for NMM were assessed in the absence of the test molecules. Fluorescence was plotted as a function of the concentration of three test molecules with constant concentrations of QB-1 (5 μM) and NMM (3 μM)(FIG. 3). The test compounds were telomestatin, QQ28, and serinodisaphyrin (see, e.g., Shin-ya et al., J. Am. Chem. Soc. 123:1262 (2001); Duan et al., Mol. Cancer Therapeutics 1:103 (2001)). IC₅₀ values were calculated from the competitive binding curves as the concentrations that yielded a 50% NMM fluorescent signal. FIG. 3 demonstrated that of the three test compounds, telomestatin interacted with the quadruplex DNA with the highest affinity, serinodisaphyrin interacted with the quadruplex DNA with intermediate affinity, and QQ28 exhibited an undetectable interaction with the quadruplex DNA. According to these results, the test molecules telomestatin and serinodisaphyrin were identified as quadruplex interacting molecules.

### Example 4

### Identification of Quadruplex Interacting Molecules by Phage Display

For display in the phage system, a random polypeptide library is encoded by PCR-based or synthesized oligonucleotide-based random library constructions as described by Andris-Widhopf et al., P9.1 or Noren et al., P19.1, Phage display: A laboratory manual, Edited by Barbas III et al.; Cold Spring Harbor Laboratory Press, Cold Spring Harbor, N.Y. The polypeptide library display and screening are conducted as described by Menendez et al., P17.1, Phage display: A laboratory manual, supra. In the screening stage of one assay format, quadruplex nucleic acids are linked to solid surfaces within wells of a microtitre plate, and phage displayed test polypeptides are added to each well. NMM also is added to each well and the fluorescent signal generated by NMM is monitored in each well. The fluorescent signal from each well is compared to fluorescent signals generated in control wells, which contain NMM and quadruplex nucleic acid without phage displayed polypeptide. Phages in wells emitting a fluorescent signal that is significantly reduced as compared to the fluorescent signal detected in control wells (e.g., half the signal or less) are recovered from each well and further subjected to enrichment, amplification, and/or sequencing steps. The sequencing step deduces a partial nucleic acid sequence that encodes a quadruplex-interacting polypeptide, and this sequence is compared to nucleic acid sequences in publicly available databases, such as GenBank (http address www.ncbi.nlm.nih.gov) to identify the full-length sequence of the polypeptide. Also, a portion or all of the partial nucleic acid sequence sometimes is utilized to synthesize a nucleic acid used to detect longer nucleic acids having a homologous sequence in nucleic acid libraries.

### Example 5

### Identification of Quadruplex Interacting Molecules by Probing Expression Libraries

A cDNA expression library (Invitrogen (San Diego, Calif.) GATEWay expression cDNA libraries) is deconvoluted into small portions and transfected into host cells, such as HeLa cells. 48 to 72 hours after transfection, the cells are lysed and the cell lysates are used as sources of expressed polypeptide targets. Alternatively, the cDNA library is engineered for expression of fusion proteins, where each polypeptide expressed by the nucleic acid library is fused to a signal peptide that facilitates secretion of the expressed polypeptides into a culture medium. Use of such fusion polypeptides negates a cell lysis step. Target polypeptides then are incubated with quadruplex-forming oligonucleotides, and NMM is added typically to a final concentration of 3 μM. In one format, polypeptide lysates/supernatant fractions are added to wells in a microtitre plate in which quadruplex nucleic acids are affixed. In another assay format, the quadruplex nucleic acids are not affixed to a solid surface of the wells. Quadruplex interacting polypeptides are identified initially in microtitre plate wells that emit a decreased fluorescent signal as compared to the fluorescent signal of NMM in control wells, where NMM and quadruplex nucleic acid are present and no expressed polypeptide is present. In wells exhibiting reduced fluorescent signals, plasmid is recovered and subjected to further amplification and sequencing steps. The sequencing step deduces a partial nucleic acid sequence that encodes the expressed polypeptide, and this sequence is compared to nucleic acid sequences in publicly available databases, such as GenBank (http address www.ncbi.nlm.nih.gov) to identify the full-length sequence of the polypeptide. Also, a portion or all of the partial nucleic acid sequence sometimes is utilized to synthesize a nucleic acid used to detect longer nucleic acids having a homologous sequence in nucleic acid libraries.

### Example 6

### Specificity of Signal Molecules for Single-Stranded DNA and Double-Stranded DNA

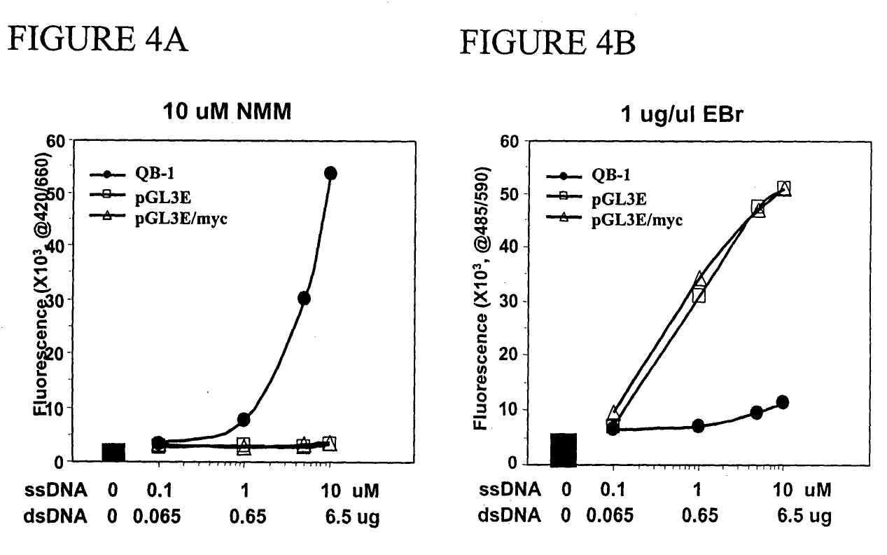

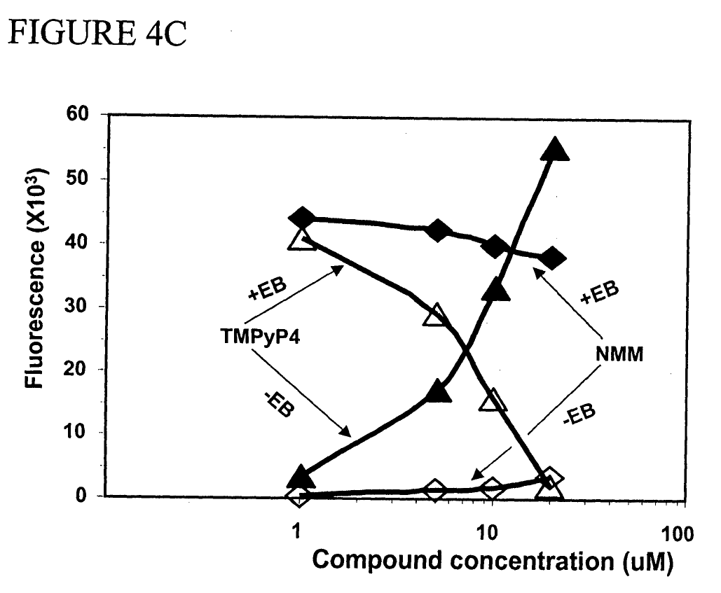

The specificity of signal molecules NMM and ethidium bromide was assessed with respect to single-stranded (ss) DNA and double-stranded (ds) DNA. Fluorescence was plotted as a function of the concentration of ss DNA QB-1 or ds DNAs pGL3E or pGL3E/myc, under conditions of 3 μM NMM (FIG. 4A) or 3 μM EB (FIG. 4B). Fluorescence of NMM was measured as described in Example 1. Fluorescence of EB was measured at an excitation wavelength of 485 nm and an emission wavelength 590 nm using a FluoStar 2000 fluorometer (BMG Labtechnologies, Durham, N.C.). Ethidium bromide (EB) was obtained from Calbiochem, San Diego, Calif. FIG. 4A demonstrated that NMM interacted with ssDNA and did not interact appreciably with ds DNA. EB, however, interacted with ds DNA and did not interact appreciably with ss DNA.

Next, the effect of EB competition with NMM or TMPyP4 for ds DNA was assessed. The assay was carried out as described in the previous paragraph, with 6.5 μg/μl pGL3, and the results were graphically compiled in FIG. 4C. FIG. 4C demonstrated that while NMM did not interact with ds DNA, which was expected in accordance with FIG. 4A, TMPyP4 did interact with ds DNA. The study also showed that EB competed with TMPyP4 for the interaction.

pGL3 was purchased from Promega, Madison, Wis. pGL3/myc included the nuclease hypersensitive element (NHE) MYC8 fragment from the proximal region of human c-MYC. pGL3/myc was constructed by cloning the MYC8 fragment from human genomic DNA (Promega, Madison, Wis.) and inserting that fragment into the multiple cloning site of the pGL3 vector via Nhe I and Bgl II digestion and ligation. The MYC8 fragment (840 bp) was cloned from human genomic DNA using two primer oligonucleotides. The sense strand primer oligonucleotide for the c-MYC regulatory region had the nucleotide sequence 5′-AGCTGCTAGCCCTGCGATGATTTATACTCA-3′ (SEQ ID NO: 36; Nhe I site is underlined), which corresponded to site 1993 in the sense strand GenBank sequence. The antisense primer had the nucleotide sequence 5′-ATCGAGATCTAGAGCCTTTCAGAGAAGCGG-3′ (SEQ ID NO: 37; Bgl II site is underlined), which corresponded to site 2833 in the sense strand GenBank sequence. The Genbank sequence was the Homo sapiens MYC gene, c-myc proto-oncogene and ORF1, Length=10996 (http address: www.ncbi.nlm.nih.gov/entrez/query.fcgi?cmd=Retrieve&db=nucleotide&list_uids=188965&dopt=GenBank). The construct was confirmed by sequencing.

### Example 7

### Secondary Methods for Identifying Quadruplex Interacting Molecules

Test nucleic acids identified as quadruplex forming nucleic acids by the assays described in Example 2 often are further confirmed for quadruplex forming function in subsequent assays. Also, test molecules identified as quadruplex interacting molecules in the assays set forth in Examples 3 and 4 often are further confirmed for quadruplex interacting activity in subsequent assays. Subsequent assays include mobility shift assays, DMS methylation protection assays, polymerase arrest assays, and transcription reporter assays.

Gel Electrophoretic Mobility Shift Assay (EMSA)

EMSA is conducted as described previously (Jin & Pike, Mol. Endocrinol. 10: 196-205 (1996)) with minor modifications. Synthetic single-stranded oligonucleotides are labeled in the 5′-terminus with T4-kinase in the presence of [α-³²P] ATP (1,000 mCi/mmol, Amersham Life science) and purified through a sephadex column. ³²P-labeled oligonucleotides (˜-30,000 cpm) then are incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25×Tris borate-EDTA buffer (0.25×TBE, 1×TBE is 89 mM Tris-borate, pH 8.0, 1 mM EDTA). The gel is dried and each band is quantified using a phosphorimager.

DMS Methylation Protection Assay

Bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoathanol:water:NaOAc (3 M); 1:6:7; v:v:v). Following ethanol precipitation (twice) and piperidine cleavage, the reactions are separated on a preparative gel (16%) and visualized on a phosphorimager.

Polymerase Arrest Assay

An example of the Taq polymerase stop assay is described in Man et al., Nucl. Acids Res. 27: 537-542 (1999), which is a modification of that used by Weitzmann et al., J. Biol. Chem. 271, 20958-20964 (1996). Briefly, a reaction mixture of template DNA (50 nM), Tris-HCl (50 mM), MgCl₂ (10 mM), DTT (0.5 mM), EDTA (0.1 mM), BSA (60 ng), and 5′-end-labeled quadruplex nucleic acid (˜18 nM) is heated to 90° C. for 5 minutes and allowed to cool to ambient temperature over 30 minutes. Taq Polymerase (1 μl) is added to the reaction mixture, and the reaction is maintained at a constant temperature for 30 minutes. Following the addition of 10 μl stop buffer (formamide (20 ml), 1 M NaOH (200 μl), 0.5 M EDTA (400 μl), and 10 mg bromophenol blue), the reactions are separated on a preparative gel (12%) and visualized on a phosphorimager. Adenine sequencing (indicated by “A” at the top of the gel) is performed using double-stranded DNA Cycle Sequencing System from Life Technologies. The general sequence for the template strands is TCCAACTATGTATAC-INSERT-TTAGCGACACGCAATTGCTATAGTGAGTCGTATTA. Bands on the gel that exhibit slower mobility are indicative of quadruplex formation.

Transcription Reporter Assay

A luciferase promoter assay described in He et al., Science 281: 1509-1512 (1998) often is utilized for the study of quadruplex formation. Specifically, a vector utilized for the assay is set forth in reference 11 of the He et al. document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the pGL3-MYC8 plasmid. Firefly and Renilla luciferase activities are assayed using the Dual Luciferase Reporter Assay System (Promega) in a 96-well plate format according to the manufacturer's protocol.

The contents of each document cited herein is incorporated by reference.

What is claimed is:

1. A method for identifying a quadruplex interacting molecule, which comprises:
contacting a test molecule and a signal molecule with a quadruplex nucleic acid in a system; and
detecting the signal produced by the signal molecule, wherein the signal produced by the signal molecule when the test molecule is present in the system and interacts with the quadruplex nucleic acid is different than the signal produced by the signal molecule when the test molecule is not present in the system;
whereby the test molecule is identified as a quadruplex interacting molecule when the signal detected in a system that includes the test molecule is different than the signal detected in a system that does not include the test molecule or includes a test molecule that does not interact with the quadruplex nucleic acid.

2. The method of claim 1, wherein the quadruplex DNA is attached to a solid support.

3. The method of claim 1, wherein the signal molecule is attached to a solid support.

4. The method of claim 1, wherein the signal molecule is a chromophore.

5. The method of claim 4, wherein the chromophore is a fluorophore.

6. The method of claim 5, wherein the fluorophore is N-methylmesoporphyrin.

7. The method of claim 1, wherein the signal that is detected is a fluorescent signal.

8. The method of claim 1, wherein the test molecule is an organic molecule or inorganic molecule having a molecular weight of 10,000 grams per mole or less.

9. The method of claim 1, wherein the test molecule is a polypeptide.

10. The method of claim 1, wherein the test molecule is a polypeptide linked to a phage.

11. The method of claim 1, wherein the test molecule is a polypeptide expressed by a microorganism transfected with a nucleic acid from an expression library.

12. The method of claim 1, wherein the test molecule and the signal molecule are contacted with the quadruplex nucleic acid simultaneously.

13. The method of claim 1, wherein the quadruplex nucleic acid comprises a nucleotide sequence selected from the group consisting of the nucleotide sequences set forth in Table 1 and Table 2.

14. A method for identifying a quadruplex forming nucleic acid, which comprises:
contacting a signal molecule with a test nucleic acid in a system, wherein the test nucleic acid is a genomic DNA fragment or complementary DNA fragment; and
detecting the fluorescent signal produced by the signal molecule, wherein the signal produced by the signal molecule when the test nucleic acid is present in the system and interacts with a quadruplex forming nucleic acid is different than the signal produced by the signal molecule when the test nucleic acid is not present in the system or the test nucleic acid does not form a quadruplex;
whereby the test nucleic acid is identified as a quadruplex forming nucleic acid when the signal detected in a system that includes the test nucleic acid is different than the signal detected in a system that does not include the test nucleic acid or includes a test nucleic acid that does not form a quadruplex.

15. The method of claim 14, wherein the quadruplex DNA is attached to a solid support.

16. The method of claim 14, wherein the signal molecule is attached to a solid support.

17. The method of claim 14, wherein the quadruplex nucleic acid comprises a nucleotide sequence selected from the group consisting of the nucleotide sequences set forth in Table 1 and Table 2.

18. The method of claim 14, wherein the signal molecule is N-methylmesoporphyrin.
